(19) United States
(12) Patent Application Publication
(10) Pub. No.: US 2017/0065781 A1
(43) Pub. Date: Mar. 9, 2017

(54) TRACHEOSTOMY TUBE ASSEMBLIES, INNER CANNULAE AND METHODS OF MANUFACTURE

(71) Applicant: SMITHS MEDICAL INTERNATIONAL LIMITED, Kent (GB)

(72) Inventors: Stephen James Field, Kent (GB); Christopher Stratton Turnbull, Kent (GB)

(73) Assignee: SMITHS MEDICAL INTERNATIONAL LIMITED, Kent (GB)

(21) Appl. No.: 15/123,009

(22) PCT Filed: Mar. 3, 2015
(86) PCT No.: PCT/GB2015/000073

§ 371 Date: Sep. 1, 2016

(30) Foreign Application Priority Data
Mar. 8, 2014 (GB) 1404219.6

(51) Int. Cl. A61M 16/04; B29C 49/04; B29C 49/42; B29C 49/48; B29C 33/42; A61M 16/08; B29C 49/14

(52) U.S. Cl. CPC A61M 16/0427; A61M 16/0434; A61M 16/0465; A61M 16/0816; B29C 49/04; B29C 49/14; B29C 49/48; B29C 33/42; B29C 49/4273; A61M 2207/10; B29L 2031/753

(57) ABSTRACT

An inner cannula (20) for a tracheostomy tube is made by extruding tubing (41) and cutting into separate lengths or preforms (42). Each preform (42) is placed in a mould (50, 51, 52) and blow moulded to expand the preform into contact with an inner surface (53, 54) of the mould which is shaped to produce a pattern on the outside of the preform of two intersecting diagonal corrugations (211, 212) that act to strengthen the blow moulded preform against lateral forces. The inner surface of the mould may also be textured so that the blow moulded preform is given a texture (213) to reduce friction with the outer tube (1).

This invention relates to methods of making an inner cannula of the kind for a tracheostomy tube assembly.

Tracheostomy tube assemblies commonly include an outer tube and an inner tube or cannula that is a removable fit within the outer tube. The inner cannula can be removed and replaced periodically to ensure that the passage through the assembly does not become blocked by secretions. This avoids the need to remove the outer tube frequently.

The inner cannula presents various problems because it must be thin walled and a close fit within the outer tube so as to provide a large bore and thereby limit the resistance to flow of gas along the assembly. It must, however, also be sufficiently stiff to be inserted in the outer tube without buckling or kinking. WO94/01156 and WO2004/101048 describe inner cannulae made of PTFE. EP1938857 describes an arrangement of tracheostomy tubes and inner cannulae where the hubs of the inner cannulae of different sizes are shaped differently so that they will only fit in the appropriate tracheostomy tube. EP2224985 describes an arrangement for attaching a hub to the shaft of an inner cannula. GB2056285 describes an inner cannula having a wall with annular corrugations and a longitudinal groove or other reinforcement member traversing at least some of the corrugations. U.S. Pat. No. 4,817,598 describes a smooth-walled inner cannula having a ring-pull formation at its rear, machine end. U.S. Pat. No. 5,119,811 describes an inner cannula with a flared patient end and formed of two layers of different materials. U.S. Pat. No. 5,386,826 describes an inner cannula with an outer helical filament or layer of low friction material. U.S. Pat. No. 5,983,895 describes an inner cannula with straight sections at opposite ends joined by an intermediate curved section. U.S. Pat. No. 6,019,753 describes an inner cannula with two elongate regions of different flexibility so that the cannula has a plane of preferential bending. U.S. Pat. No. 6,019,753 describes an inner cannula having a shaft formed with slots to make it more flexible, the slots being covered by an outer thin sheath. U.S. Pat. No. 6,135,110 describes a curved inner cannula that is retained with the outer tube by means of a rotatable spring fitting.

It is an object of the present invention to provide an alternative inner cannula, a tracheostomy tube assembly including an inner cannula and a method of making an inner cannula.

According to one aspect of the present invention there is provided a method of the above-specified kind, characterised in that the method includes the steps of forming a tubular plastics preform and subsequently heating, stretching and blow moulding the preform to an increased diameter in an external mould, the mould having an inner surface provided with a pattern of surface formations such that the preform is blow moulded externally with a corresponding pattern of surface formations, and that the surface formations are adapted to increase the strength of the moulded article against lateral forces.

The pattern of surface formations is preferably a pattern of intersecting diagonal corrugations. Alternatively, the pattern of surface formations includes circumferential formations such as annular or helical formations. The inner surface of the mould may be textured to produce a corresponding texture on the external surface of the moulded article arranged to reduce friction. The inner surface of the mould could also include one or more axially-extending surface formations so that the moulded article is formed with corresponding axial formations effective to increase the compressive strength of the article. The method may include the additional step of attaching a hub to one end of the moulded article. Alternatively, the method may include forming a hub integrally with the tubular part of the cannula.

According to another aspect of the present invention there is provided an inner cannula made by a method according to the above one aspect of the present invention.

According to a further aspect of the present invention there is provided an inner cannula with a shaft of a plastics material having on its external surface a pattern of corrugations to reinforce the shaft primarily against lateral forces, characterised in that the pattern of corrugations includes at least two sets of parallel corrugations that are inclined relative to one another so that they intersect at an angle to form a generally diamond-shape pattern.

The cannula may additionally include a texture on its external surface arranged to reduce friction.

According to a fourth aspect of the present invention there is provided a tracheostomy tube assembly including an outer tracheostomy tube and an inner cannula according to the above other or further aspect of the present invention, the inner cannula being inserted within the outer tube and being removable therefrom.

A method of making an inner cannula for a tracheostomy tube assembly in accordance with the present invention, will now be described, by way of example, with reference to the accompanying drawings, in which:

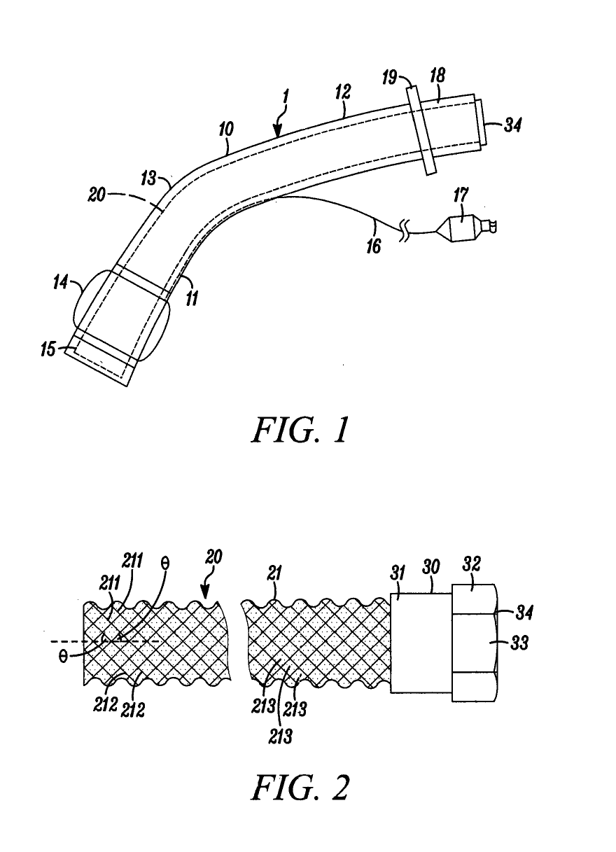

FIG. 1 is a side elevation view of a tracheostomy tube assembly including an inner cannula;

FIG. 2 is a side elevation view of the inner cannula;

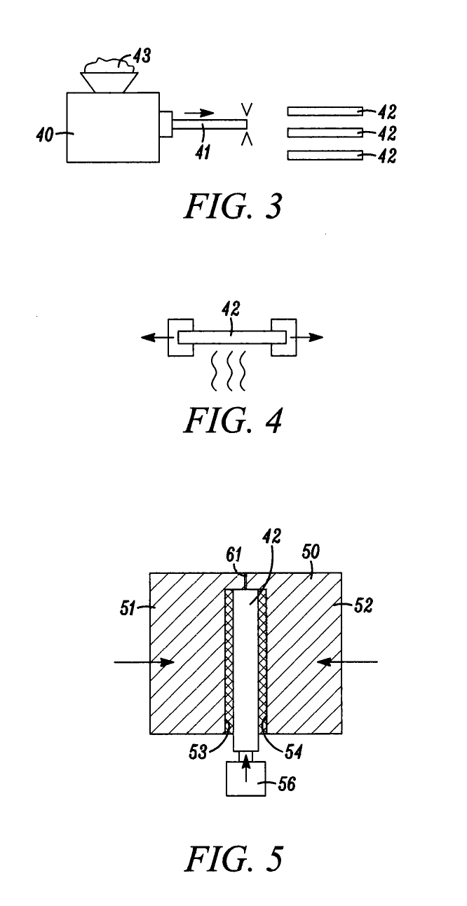

FIGS. 3 to 6 show successive steps in the process of manufacturing the inner cannula; and

FIG. 7 illustrates a modification of the manufacturing process.

With reference first to FIG. 1, the tracheostomy tube assembly comprises an outer tracheostomy tube 1 and a removable inner cannula 20 inserted within the outer tube. The outer tube I has a shaft 10 with a substantially straight forward or patient end section 11, a substantially straight rear or machine end section 12 and a curved intermediate section 13 linking the forward and rear sections. An inflatable sealing cuff 14 embraces the forward section 11 close to the patient end 15 of the tube 1, the cuff being inflated via an inflation lumen 16 and a combined connector and inflation indicator 17. At its rear end the outer tube 1 has a hub 18 and flange 19 to which a retaining tape can be fastened for securing the tube with the patient's neck. The inside of the hub 18 provides a female 15 mm connector and is formed with keying flats (not shown), of the kind described in EP1938857, adapted to prevent full insertion of an inner cannula of the wrong size. The outer tube 1 could have an internal diameter between about 2 mm and 10 mm, and its length could be between 60 mm and 200 mm. Other shape tracheostomy tubes could be used.

With reference now also to FIG. 2, the inner cannula 20 comprises a shaft 21 of circular section attached at its rear or machine end with a hub 30. The hub 30 has a forward, patient end portion 31 shaped to fit into a 15 mm connector. To the rear of the forward portion 31 the hub 30 has a keying portion 32 provided with flats 33 of the kind described in EP1938857 adapted to fit with corresponding formations in the hub 18 of the outer tube 1. At its rear end the inner cannula 20 has a ring-pull formation 34 of the kind described in U.S. Pat. No. 4,817,598, which facilitates removal of the inner cannula from the outer tube 1 after use.

The shaft 21 of the inner cannula 20 has a diamond pattern formed on its outer surface by two sets of straight, parallel corrugations 211 and 212 extending at an angle θ inclined to the axis of the cannula (typically of around 30°) in opposite senses. In this way, the two sets of corrugations 211 and 212 intersect one another diagonally at an angle of about 60° to give a diamond pattern of corrugations. The corrugations 211 and 212 are formed through the thickness of the wall of the shaft 21 so that the corrugations are present on both the internal and external surfaces, although on the inner surface this pattern is less sharply defined. The corrugations may be smoothly rounded (of sine wave shape) or may be more angular, of triangular profile. Other profiles are also possible. This arrangement of corrugations 211 and 212, as well as giving the cannula 20 strength against lateral/radial crushing forces, also increases the axial stiffness of the cannula so that it is less prone to being compressed along its length and thereby retains its desired length during insertion within the outer tracheal tube 1.

Instead of the diamond pattern of corrugations it would be possible to have conventional circumferential corrugations, such as of annular or helical configuration. The corrugations may be intersected by one or more (usually two) longitudinally-extending ridges or channels formed along the surface to enhance axial stiffness. Such longitudinal reinforcements may be more necessary with circumferential corrugations than with the diamond pattern of corrugations. Other patterns of surface formations could be used, such as star shapes or H shapes. Preferably these shapes would be arranged such that a deformation in the longitudinal direction is accommodated by flexing of a spring section of the pattern, so that the tube can be bent without kinking.

The external surface of the shaft 21 also has a texture 213 to reduce friction with the inside of the outer tube 1. The texture 213 may take the form of a frosted surface, a dimpled surface or a similar surface with an array of projections above the surface, which projections are much smaller than the depth of the corrugations 211 and 212. The texture 213 only appears on the outside of the shaft 21 and not on the inside.

The inner cannula 20 is made by a stretch blow moulding technique, as shown in FIGS. 3 to 6. An extruder machine 40, shown in FIG. 3, extrudes a length of tubing 41 from a plastics material 43 suitable for use in a stretch blow moulding technique, such as polyethylene, polypropylene, polyester or other crystal or semi-crystal materials. The tubing 41 has an external diameter less than that required for the inner cannula 20 itself. The tubing 41 is extruded continuously and is then trimmed to form preforms 42 of suitable length slightly longer than required for the final inner cannula 20. These preforms could simply be a single layer or they could be a laminate of two, three or more layers of different materials. For example, the outer layer could be of a material having a low friction and an inner layer could be of a material with a high kink resistance. These multiple layers may be achieved during the extrusion process by coextrusion or they could be made by subsequently coating the extruded component, such as by dipping. The preform could alternatively be made in other ways, such as by a co-injection process. The preforms or lengths of tubing 42 are then gripped at opposite ends and subjected to heat while at the same time applying oppositely-directed axial forces to each end so as to stretch the tubing axially slightly, as illustrated in FIG. 4. The stretching treatment causes an increase in the axial alignment of the molecules in the tubing 42. Alternatively, the preforms could be stretched using a hollow mandrel inserted in the preforms that is subsequently used as a conduit for blowing gas in the blow moulding step of the process. Alternatively, the stretching operation could be carried out while the tubing was mounted within a blow moulding tool.

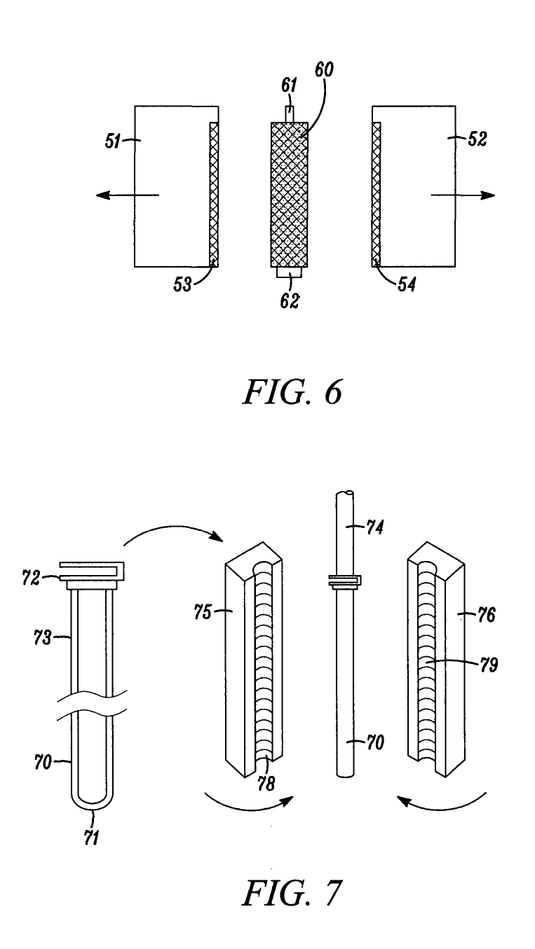

The stretched length of tubing 42 is warmed using infra-red or by contact heating and is then placed in an external blow moulding tool 50, as shown in FIG. 5. The blow moulding tool 50 is in two parts 51 and 52 each of which has a semi-cylindrical mould cavity 53 and 54 the dimensions and configuration of which correspond to the desired final shape of the shaft 21 of the inner cannula 20. The surface of the two mould cavities 53 and 54 is configured with the inverse or negative pattern of corrugations to that desired to be formed on the outside of the inner cannula 20 such that concave surfaces on the inside of the mould cavity result in corresponding convex surfaces on the outside of the shaft 21 of the inner cannula. The two parts 51 and 52 of the mould tool 50 are moved together to clamp off the upper end 61 of the length of tubing 42. The lower end 62 of the length of tubing 42 is connected to a source 56 of high pressure air or other gas. The tubing 42 is expanded outwardly into contact with the surface of the cavity 53, 54 so that the material of the tubing is pressed into and around the recesses and other surface formations in the cavity. The diameter of the tubing is, therefore, increased and its wall thickness is reduced. The inner surface of the expanded tubing 42 will have some of the pattern on the outer surface but it will not be in such sharp definition. The mould tool 50 is cooled so as to cool the expanded preform when it contacts the surface of the cavities 53 and 54. The two parts 51 and 52 of the tool 50 are then separated, as shown in FIG. 6, and the blow moulded component 60 is removed. The upper end 61 of the component 60 is then cut to remove the pinched closed upper end. The lower end 62 is also cut to remove the small length of the component 60 outside the mould tool 50 that has not been expanded and to trim the component to the desired length. The machine end hub 30 is then attached to one end of the component 60 to complete the inner cannula 20.

Instead of attaching the hub to the blow moulded component after this has been formed, it would be possible to make the hub integrally with the shaft, in one piece, as illustrated in FIG. 7. This shows a moulded preform 70 with a closed lower end 71 and with a hub 72 moulded integrally with the upper end of the tubular part 73 of the preform. A mandrel and blow pin 74 is inserted in the upper end of the preform 70, which is then enclosed between two parts 75 and 76 of a mould tool 77 so that the tubular part 73 can be expanded by gas pressure from the blow pin into contact with the patterned surface of the mould cavities 78 and 79.

The inner cannula 20 with the diamond pattern of corrugations could be formed in other ways than by the stretch blow moulding technique described above.

Instead of forming the entire pattern of surface formations by contact with the inside of a blow mould tool, it would be possible for the preform to be formed with a part pattern before the blow moulding process, such as by extruding this into the outer surface or forming by some other moulding technique.

1-12. (canceled)

13. A method of making an inner cannula for a tracheostomy tube assembly, characterized in that the method includes forming a tubular plastics preform and subsequently heating, stretching and blow molding the preform to an increased diameter in an external mould, the mould having an inner surface provided with a pattern of surface formations such that the preform is blow molded externally with a corresponding pattern of surface formations, and that the surface formations are adapted to increase the strength of the molded article against lateral forces.

14. A method according to claim 13, characterized in that the pattern of surface formations is a pattern of intersecting diagonal corrugations.

15. A method according to claim 13, characterized in that pattern of surface formations includes circumferential corrugations.

16. A method according to claim 15, characterized in that the circumferential corrugations are annular or helical formations.

17. A method according to claim 13, characterized in that the inner surface of the mould is textured to produce a corresponding texture on the external surface of the blow molded article arranged to reduce friction.

18. A method according to claim 13, characterized in that the inner surface of the mould also includes one or more axially-extending surface formations so that the molded article is formed with one or more corresponding axial formations effective to increase the compressive strength of the article.

19. A method according to claim 13, characterized in that the method includes attaching a hub to one end of the blow molded article.

20. A method according to claim 13, characterized in that the method includes forming a hub integrally with the tubular part of the cannula.

21. An inner cannula made from a tubular plastic preform subsequently heated, stretched and blow molded to an increased diameter in an external mold having an inner surface provided with a pattern of surface formations such that the preform is blow molded externally with a corresponding pattern of surface formations that are adapted to increase the strength of the section against lateral forces.

22. An inner cannula with a shaft of a plastics material having on its external surface a pattern of corrugations to reinforce the shaft primarily against lateral forces, characterized in that the pattern of corrugations includes at least two sets of parallel corrugations that are inclined relative to one another so that they intersect at an angle to form a generally diamond-shape pattern.

23. An inner cannula according to claim 22, characterized in that the cannula additionally includes a texture on its external surface arranged to reduce friction.

24. A tracheostomy tube assembly including an outer tube and an inner cannula having a section formed from a tubular plastic preform subsequently heated, stretched and blow molded to an increased diameter in an external mold having an inner surface provided with a pattern of surface formations such that the preform is blow molded externally with a corresponding pattern of surface formations that are adapted to increase the strength of the section against lateral forces, wherein the inner cannula is inserted within the outer tube and is removable therefrom.
